(19) United States
(12) Patent Application Publication
(10) Pub. No.: US 2003/0186277 A1
(43) Pub. Date: Oct. 2, 2003

(54) Diagnosis of known genetic parameters within the mhc

(76) Inventors: Alexander Olek, Berlin (DE); Christian Piepenbrock, Berlin (DE); Kurt Berlin, Stahnsdorf (DE)

Correspondence Address: KRIEGSMAN & KRIEGSMAN, 665 FRANKLIN STREET, FRAMINGHAM, MA, 01702, US

(21) Appl. No.: 10/312,841

(22) PCT Filed: Jun. 29, 2001
(86) PCT No.: PCT/IB01/01501

(30) Foreign Application Priority Data
Jun. 30, 2000 (DE) 100 32 529.7

(51) Int. Cl. C12Q 1/68; C07H 21/04; C07K 14/00

(52) U.S. Cl. 435/6; 530/350; 536/24.3

(57) ABSTRACT

The present invention describes nucleic acids for the diagnosis of a set of genetic parameters within the major histocompatibility complex (MHC), comprising a segment that is inversely complementary or identical to a chemically pretreated genomic DNA and that is at least 20 base pairs long as well as a set of oligomer probes (oligonucleotides and/or PNA oligomers), which serve for the detection of the cytosine methylation state in nucleic acids. These probes are particularly suitable for the diagnosis of genetic parameters within the MHC.

The present invention concerns nucleic acids, oligonucleotides, PNA oligomers and a method for the diagnosis of important genetic parameters within the major histocompatibility complex (MHC).

The levels of observation that have been well studied in molecular biology according to developments in methods in recent years include the genes themselves, the transcription of these genes into RNA and the translation to proteins therefrom. During the course of development of an individual, which gene is turned on and how the activation and inhibition of certain genes in certain cells and tissues are controlled can be correlated with the extent and nature of the methylation of the genes or of the genome. In this regard, pathogenic states are expressed by a modified methylation pattern of individual genes or of the genome.

The major histocompatibility complex (MHC) describes a group of genes with immunological and non-immunological functions and is found in all vertebrates (“Both man & bird & beast”: comparative organization of MHC genes. 1995, Trowsdale J, Immunogenetics; 41: 1-17; Evolving views of the major histocompatibility complex. 1997, Gruen J R and Weissman S M, Blood; 90: 4252-4265). In humans, it extends over a region of 3.6 million base pairs on the short arm of chromosome 6 (6p21.3) and basically participates in the immune response. It has been completely sequenced, it is highly polymorphic and it has the highest gene density in the entire human genome. Thus, of the total 3500 genes estimated on chromosome 6, 224 identified gene loci are ascribed to the MHC, which means that 3 times as many genes are localized in the region of the MHC as would be expected on the basis of its size. The MHC is subdivided into three regions: class 1, 2 and 3. All genes of class 1 are between 3 and 6 kb in size. They are present on each cell and are denoted transplantation antigens, i.e., they are responsible for the rejection of foreign tissue. Genes of class 2 are between 4 and 11 kb in size. The gene products participate in the interaction between cells which are required for the immune response. Class 3 has the highest gene density. Genes that are not involved in the immune system as well as complement factors which interact with antibody-antigen complexes as components of the serum are both localized here.

The primary immunological function of MHC molecules consists of binding antigenic peptides to the surfaces of cells; this serves for the recognition of antigen-specific T-cell receptors of lymphocytes. T-cells are important in that they are closely associated with intracellular infections and tumors. Since MHC molecules play a central role in the regulation of the immune response, they probably also have a decisive role in the monitoring of and susceptibility to diseases. It is assumed that the MHC is associated with genetic disorders such as rheumatoid arthritis (The association of HLA-DM genes with rheumatoid arthritis in Eastern France. 2000, Toussirot E et al., Hum Immunol; 61(3):303-308), Diabetes (In vivo evidence for the contribution of human histocompatibility leukocyte antigen (HLA)-DQ molecules to the development of diabetes. 2000, Wen L et al., J Exp Med; 191(1):97-104), particularly type I diabetes (The aetiology of Type I diabetes. 1999, Chowdhury T A, Mijovic C H, Barnett A H, Baillieres Clin Endocrinol Metab.; 13(2): 181-195), and insulin-dependent diabetes mellitus (IDDM) (Identification of a new susceptibility locus for insulin-dependent diabetes mellitus by ancestral haplotype congenic mapping. 1995, Ikegami H, Makino S, Yamoto E, Kawaguchi Y, Ueda H, Sakamoto T, Takekawa K, Ogihara T, J Clin Invest.; 96(4):1936-1942), heredeitary hemochromotosis (Haemochromatosis in the new millennium. 2000, Powell L W et al., J Hepatol;32(1 Suppl):48-62), particularly genetic hemochromatosis (GH) (HFE codon 63/282 (H63D/C282Y) dimorphism in German patients with genetic hemochromatosis. 1998, Gottschalk R, Seidl C, Loffler T, Seifried E, Hoelzer D, Kaltwasser J P, Tissue Antigens; 51 (3):270-275) and the mild form of hemochromatosis (HFE mutations analysis in 711 hemochromatosis probands: evidence for S65C implication in mild form of hemochromatosis. 1999, Mura C, Raguenes O, Ferec C, Blood; 93(8):2502-2505), schizophrenia (Schizophrenia, rheumatoid arthritis and natural resistance genes. 1997, Rubinstein G, Schizophr Res; 25(3):177-181), HIV (The human immunodeficiency virus type 1 (HIV-1) Vpu protein interferes with an early step in the biosynthesis of major histocompatibility complex (MHC) class I molecules. 1997, Kerkau T et al., Exp Med; 185(7):1295-1305), Myositis (Mapping of a candidate region for susceptibility to inclusion body myositis in the human major histocompatibility complex. 1999, Kok C C et al. Immunogenetics; 49(6):508-516), psoriasis (Localization of Psoriasis-Susceptibility Locus PSORS1 to a 60-kb Interval Telomeric to HLA-C. 2000; Nair R P et al., Am J Hum Genet Jun; 66(6):1833-1844), systemic lupus erythematosus (The genetics of systemic lupus erythematodes. 1999, Lindqvist A K, Alarcon-Riquelme M E; Scand J Immunol; 50(6):562-571), IgA nephropathy (Evidence for genetic factors in the development and progression of IgA nephropathy. 2000, Hsu Si et al., Kidney Int; 57(5):1818-1835. Review), hypertension (Possible influence of genes located an chromosome 6 within or near to the major histocompatibility complex on development of essential hypertension. 2000, Vidan-Jeras B et al., Pflugers Arch; 439 (3 Suppl):R6062), Behcet disease (The critical region for Behcet disease in the human major histocompatibility complex is reduced to a 46-kb segment centromeric of HLA-B, by association analysis using refined microsatellite mapping. 1999, Ota M et al., Am J Hum Genet; 64(5):1406-1410), Gee-Heubner-Herter-Thaysen disease (CTLA-4 gene polymorphism is associated with predisposition to coeliac disease. 1998, Djilali-Saiah I et al., Gut; 43(2):187-189), Myasthenia gravis, spondyloarthropathy (Genes in the spondyloarthropathies. 1998, Wordsworth P., Rheum Dis Clin North Am; 24(4):845-863. Review), tuberculosis (Differential T cell responses to Mycobacterium tuberculosis ESAT6 in tuberculosis patients and healthy donors. 1998, Ulrichs T et al., Eur J Immunol; 28(12):3949-3958), hypertrophic cardiomyopathy (Mutations in the cardiac troponin I gene associated with hypertrophic cardiomyopathy. 1997, Kimura A., Nat. Genet. 16(4):379-382), Basedow's [Graves'] disease (Iodide, cytokines and TSH-receptor expression in Graves' disease. 1996, Schuppert et al., Exp Clin Endocrinol Diabetes. 104 (Suppl 4:68-74), juvenile rheumatoid arthritis (HLA and T cell receptor polymorphisms in pauciarticular-onset juvenile rheumatoid arthritis. 1991, Nepom B S, Arthritis Rheum. 34(10): 1260-1267), epilepsy, idiopathic generalized epilepsy (The phenotypic spectrum related to the human epilepsy susceptibility gene EJM1. 1995, Sander T, Ann Neurol; 38(2):210-217), juvenile myoclonic epilepsy (Refined mapping of the epilepsy susceptibility locus EJM1 on chromosome 6. 1997, Sander T, Neurology. 49(3):842-847), Takayasu disease, multiple immunopathological disorders (The genetic basis for the association of the 8.1 ancestral haplotype (A1,B8,DR3) with multiple immunopathological diseases. 1999, Price P, Immunol Rev. 167: 257-274), head and neck cancer (Influence of tumour necrosis factor microsatellite polymorphisms on susceptibility to head and neck cancer. 1998; susceptibility to leprosy in humans. 1996; Lagrange P H et al., Acta Leprol; 10(1):11-27), malaria (Genetic susceptibility to malaria and other infectious diseases: from the MHC to the whole genome. 1996; Hill A V, Parasitology; 112 Suppl: 75-84, Genetic epidemiology in the study of susceptibility/resistance to malaria in the human population. 1999; Abel L, Bull Soc Pathol Exot;92(4):256-60), leishmaniasis (Genetics of host resistance and susceptibility to intramacrophage pathogens: a study of multicase families of tuberculosis, leprosy and leishmaniasis in northeastern Brazil. 1998; Blackwell J M, Int J Parasitol; 28(1):21-28), sarcoidosis (Analysis of MHC encoded antigen-processing genes TAP1 and TAP2 polymorphisms in sarcoidosis. 1999; Foley P J et al., Am J Respir Crit Care Med; 160(3):1009-1014), multiple sclerosis (DRB1-DQA1-DQB1 loci and multiple sclerosis predisposition in the Sardinian population. 1998; Marrosu M G et al., Hum Mol Genet; 7(8):1235-1237), primary biliary cirrhosis (Genetic susceptibility to primary biliary cirrhosis. 1999; Agarwal K et al., Eur J Gastroenterol Hepatol. June;11(6):603-606), nephritis (Genetic susceptibility to lupus nephritis. 1998; Tsao B P, Lupus; 7(9):585-590) and many other disorders.

There are investigations that demonstrate that the expression of MHC genes is coupled to the methylation of CpG dinucleotides, which can negatively influence transcription. This can be done directly in that the transcription factors do not bind with the DNA, or indirectly, by repressor molecules, which bind to methylated CpGs (How does DNA methylation repress transcription?, 1997; Kass S U et al., Trends Genet; 11:444-449).

Different results demonstrate the relationship between immunological disorders and methylation. The relationship between the expression of HLA-DR antigens and the methylation of the HLA-DR alpha gene was investigated in systemic lupus erythematosus, a generalized autoimmune disease (Low expression of human histocompatibility leukocyte antigen-DR is associated with hypermethylation of human histocompatibility leukocyte antigen-DR alpha gene regions in B cells from patients with systemic lupus erythematosus. 1985; Sano H et al., J Clin Invest, 76(4):1314-1322). The participation of the DNA methylation in aberrant MHC class II gene expression was investigated in patients with MHC class II deficiency syndrome (The MHC class II deficiency syndrome: heterogeneity at the level of the response to 5-azadeoxycytidine. 1990; Lambert M et al., Res Immunol. 141(2):129-140). Evidence of the regulation of MHC genes based on an epigenetic mechanism has been furnished (Methylation of class II trans-activator promoter IV: a novel mechanism of MHC class II gene control. 2000, Morris A C et al., J Immunol; 164(8):4143-4149). The expression of MHC genes is inhibited if transcription factors do not bind to the class 2 trans activator (CIITA) promotor. The inhibition here is based on the methylation of CpG dinucleotides in the pIV promotor; in contrast, inhibition of the methylation leads to a re-expression of the CIITA gene. In addition, the expression could not be stimulated in a transient transfection experiment by methylated pIV DNA. These results demonstrate an epigenetic regulation of CIITA and further allow the conclusion that this epigenetic control applies in principle to MHC class II genes.

5-Methylcytosine is the most frequent covalently modified base in the DNA of eukaryotic cells. For example, it plays a role in the regulation of transcription, in genetic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

A relatively new method that in the meantime has become the most widely used method for investigating DNA for 5-methylcytosine is based on the specific reaction of bisulfite with cytosine, which, after subsequent alkaline hydrolysis, is then converted to uracil, which corresponds in its base-pairing behavior to thymidine. In contrast, 5-methylcytosine is not modified under these conditions. Thus, the original DNA is converted so that methylcytosine, which originally cannot be distinguished from cytosine by its hybridization behavior, can now be detected by “standard” molecular biology techniques as the only remaining cytosine, for example, by amplification and hybridization or sequencing. All of these techniques are based on base pairing, which is now completely utilized. The prior art, which concerns sensitivity, is defined by a method that incorporates the DNA to be investigated in an agarose matrix, so that the diffusion and renaturation of the DNA is prevented (bisulfite reacts only on single-stranded DNA) and all precipitation and purification steps are replaced by rapid dialysis (Olek, A. et al., Nucl. Acids Res. 1996, 24, 5064-5066). Individual cells can be investigated by this method, which illustrates the potential of the method. Of course, up until now, only individual regions of up to approximately 3000 base pairs long have been investigated; a global investigation of cells for thousands of possible methylation analyses is not possible. Of course, this method also cannot reliably analyze very small fragments of small quantities of sample. These are lost despite the protection from diffusion through the matrix.

An overview of other known possibilities for detecting 5-methylcytosines can be derived from the following review article: Rein, T., DePamphilis, M. L., Zorbas, H., Nucleic Acids Res. 1998, 26, 2255.

With few exceptions the bisulfite technique has been previously applied only in research (e.g. Zechnigk, M. et al., Eur. J. Hum. Gen. 1997, 5, 94-98). However, short, specific segments of a known gene have always been amplified after a bisulfite treatment and either competely sequenced (Olek, A. and Walter, J., Nat. Genet. 1997, 17, 275-276) or individual cytosine position are detected by a “primer extension reaction” (Gonzalgo, M. L. and Jones, P. A., Nucl. Acids Res. 1997, 25, 2529-2531, WO Patent 95 00669) or an enzymatic step (Xiong, Z. and Laird, P. W., Nucl. Acids. Res. 1997, 25, 2532-2534). Detection has also been described by hybridization (Olek et al., WO 99 28498).

Other publications which are concerned with the application of the bisulfite technique for the detection of methylation in the case of individual genes are: Xiong, Z. and Laird, P. W. (1997), Nucl. Acids Res. 25, 2532; Gonzalgo, M. L. and Jones, P. A. (1997), Nucl. Acids Res. 25, 2529; Grigg, S. and Clark, S. (1994), Bioassays 16,=431; Zeschnik, M. et al. (1997), Human Molecular Genetics 6, 387; Teil, R. et al. (1994), Nucl. Acids Res. 22, 695; Martin, V. et al. (1995), Gene 157, 261; WO 97 46705, WO 95 15373 and WO 45560.

Matrix-assisted laser desorptions/ionization mass spectrometry (MALDI-TOF) is a very powerful development for the analysis of biomolecules (Karas, M. and Hillenkamp, F. (1988), Laser desorption ionization of proteins with molecular masses exceeding 10000 daltons. Anal. Chem. 60: 2299-2301). An analyte is embedded in a light-absorbing matrix. The matrix is vaporized by a short laser pulse and the analyte molecule is thus transported unfragmented into the gaseous phase. The analyte is ionized by collisions with matrix molecules. An applied voltage accelerates the ions in a field-free flight tube. Ions are accelerated to varying degrees based on their different masses. Smaller ions reach the detector sooner than larger ones.

MALDI-TOF spectroscopy is excellently suitable for the analysis of peptides and proteins. The analysis of nucleic acids is somewhat more difficult (Gut, I. G. and Beck, S. (1995)), DNA and Matrix Assisted Laser Desorption Ionization Mass Spectrometry. Molecular Biology: Current Innovations and Future Trends 1: 147157.) For nucleic acids, the sensitivity is approximately 100 times poorer than for peptides and decreases overproportionally with increasing fragment size. For nucleid acids, which have a multiply negatively charged backbone, the ionization process via the matrix is essentially less efficient. The selection of the matrix plays an imminently important role in MALDI-TOF spectroscopy. Several very powerful matrices which produce a very fine crystallization have been found for the desorption of peptides. In the meantime, however, several claimed matrices for DNA have not reduced the difference in sensitivity. The difference in sensitivity, however, can be reduced by chemically modifying the DNA so that it is more like a peptide.

Phosphorothioate nucleic acids, in which the usual phosphates of the backbone are substituted by thiophosphates, can be converted into a neutrally charged DNA by simple alkylation chemistry (Gut, I. G. and Beck, S. (1995), A procedure for selective DNA alkylation and detection by mass spectrometry. Nucleic Acids Res. 23: 1367-1373). The coupling of a charge tag to this modified DNA results in an increase in sensitivity by the same amount as is found for peptides. Another advantage of charge tagging is the increased stability of the analysis in the presence of impurities that make the detection of unmodified substrates very difficult.

Genomic DNA is obtained from DNA of cells, tissue or other test samples by standard methods. This standard methodology is found in references such as Fritsch and Maniatis, eds, Molecular Cloning: A Laboratory Manual, 1989.

The object of the present invention is to present oligonucleotides and/or PNA oligomers for the detection of cytosine methylations as well as a method which is particularly suitable for the diagnosis of genetic and epigenetic parameters within the MHC.

The object is solved by nucleic acids for the diagnosis of a set of genetic parameters within the major histocompatibility complexes (MHC), comprising a segment which is at least 20 base pairs long that is inversely complementary or identical to the chemically pretreated genomic DNA according to SEQ ID-NO:1 or SEQ ID-NO:2.

One subject of the present invention is oligonucleotides for the detection of the cytosine methylation state in pretreated genomic DNA, each [oligonucleotide] comprising at least one base sequence with a length of at least 13 nucleotides, which is inversely complementary or identical to a segment of the base sequence according to SEQ ID-NO:1 or SEQ ID-NO:2, which contains at least one CpG dinucleotide.

It is preferred that the cytosine of the CpG dinucleotide is the 5^{th} to the 9^{th} nucleotide from the 5′ end of the 13-mer.

It is further preferred according to the invention that an oligonucleotide is present for each of the CpG dinucleotides from one of the [sequences] SEQ ID-NO:1 or SEQ ID-NO:2.

One subject of the present invention is PNA (peptide nucleic acid) oligomers for the detection of the cytosine methylation state in chemically pretreated genomic DNA, each [oligomer] comprising at least one PNA base sequence with a length of at least 9 nucleotides, which is inversely complementary or identical to a segment of the base sequence according to SEQ ID-NO:1 or SEQ ID-NO:2, which contains at least one CpG dinucleotide. It is preferred according to the invention that the cytosine of the CpG dinucleotide is the 4^{th} to the 6^{th} nucleotide from the 5′ end of the 9-mer. It is further preferred according to the invention that an oligonucleotide^{★} is present for each of the CpG dinucleotides from a base sequence according to SEQ ID-NO:1 or SEQ ID-NO:2. ^{★}sic; PNA oligomer?—Trans. Note.

The subject of the present invention is a set of oligomer probes for the detection of the cytosine methylation state and/or of single nucleotide polymorphisms in chemically pretreated genomic DNA, comprising at least 10 of the oligonucleotide or PNA sequences according to the invention.

The subject of the present invention is also an arrangement of different oligonucleotide and/or PNA oligomer sequences according to the invention, wherein these are bound to defined sites of a solid phase. It is preferred that these are arranged on a planar solid phase in the form of a rectangular or hexagonal lattice.

The subject of the present invention is a set of primer oligonucleotides comprising at least two oligonucleotides, each of which has a sequence that is at least 18 base pairs long and that corresponds to or is inversely complementary to the base sequences according to SEQ ID-NO:1 or SEQ ID-NO:2. It is preferable according to the invention that these do not contain a CpG dinucleotide. It is further preferred according to the invention that at least one primer is bound to a solid phase.

Another particularly preferred subject of the present invention is the use of the nucleic acids, oligonucleotides or PNA oligomers according to the invention for the diagnosis of autoimmune diseases, for the diagnosis of rheumatoid arthritis, for the diagnosis of diabetes, particularly preferred for the diagnosis of diabetes of type I diabetes or of insulin-dependent diabeted mellitus (IDDM), for the diagnosis of hereditary hemochromatosis, particularly preferred for the diagnosis of genetic hemochromatosis (GH) or the mild form of hemochromatosis, for the diagnosis of schizophrenia, for the diagnosis of multiple sclerosis, for the diagnosis of systemic lupus erythematosus, for the diagnosis of sarcoidosis, for the diagnosis of primary biliary cirrhosis, for the diagnosis of myositis, for the diagnosis of psoriasis, for the diagnosis of nephritis, for the diagnosis of cancer, particularly of head or neck cancer, for the diagnosis of IgA nephropathy, for the diagnosis of hypertension, for the diagnosis of Behcet's disease, for the diagnosis of GeeHeubner-Herter-Thaysen disease (coeliac disease), for the diagnosis of myasthenia gravis, for the diagnosis of spondyloarthropathy, for the diagnosis of tuberculosis, for the diagnosis of hypertrophic cardiomyopathy, for the diagnosis of Basedow's [Graves'] disease, for the diagnosis of juvenile rheumatoid arthritis, for the diagnosis of epilepsy, preferably idiopathic, generalized epilepsy or juvenile myoclonic epilepsy, for the diagnosis of Takayasu disease, for the diagnosis of multiple immunopathological diseases, for the diagnosis of susceptibility to leprosy, for the diagnosis of susceptibility to malaria and/or for the diagnosis of susceptibility to leishmaniasis, by analyis of methylation patterns within the MHC.

Another subject is also the use of the nucleic acids according to SEQ ID-NO:1 or SEQ ID-NO:2 according to the invention for the diagnosis of important genetic parameters within the MHC.

Another subject of the present invention is a method for the diagnosis of important genetic parameters within the MHC by analysis of cytosine methylations in sets of oligonucleotides or PNA oligomers according to one of the preceding claims^{★}, further characterized in that the following steps are conducted:

a) In a genomic DNA sample, cytosine bases that are not methylated at the 5-position are converted by chemical treatment to uracil, thymidine or another base that is unlike cytosine in its hybridization behavior;

b) fragments of this chemically pretreated genomic DNA are amplified with the use of sets of primer oligonucleotides according to the invention and a polymerase;

c) the amplified products are hybridized to a set of oligonucleotides or PNA probes according to the invention;

d) the hybridized amplified products are detected and visualized.

^{★}sic—Trans. Note.

It is preferred according to the invention that more than ten different fragments, which are 100-2000 base pairs long, are amplified. It is further preferred that the chemical treatment is conducted by means of a solution of a bisulfite, hydrogen sulfite or disulfite. It is also particularly preferred that the polymerase is a heat-stable DNA polymerase.

It is further preferred according to the invention that the amplification is conducted by means of a polymerase chain reaction (PCR). It is also preferred that labels that are introduced on the amplified products at each position of the solid phase where an oligonucleotide sequence is found can be identified. It is further particularly preferred according to the invention that an arrangement according to the invention is used and that the solid-phase surface is comprised of silicon, glass, polystyrene, aluminum, steel, iron, copper, nickel, silver or gold. It is additionally preferred that the amplification of several DNA segments is conducted in one reaction vessel. It is also preferred according to the invention that the labels of the amplified products are fluorescence labels. It is also preferred that the labels of the amplified products are radionuclides. It is also preferred that the labels of the amplified products are removable molecular fragments with typical mass which are detected in a mass spectrometer. It is particularly preferred according to the invention that the amplified products or fragments of the amplified products are detected in the mass spectrometer. For this purpose, it is advantageous according to the invention that the generated fragments have a single positive or negative net charge for better detectability in the mass spectrometer.

It is most particularly preferred according to the invention that detection and visualization are conducted by means of matrix-assisted laser desorption/ionization mass spectrometry (MALDI) or by means of electrospray mass spectrometry (ESI).

The method is preferred wherein the genomic DNA has been obtained from a DNA sample, whereby sources for DNA include, e.g., cell lines, biopsies, blood, sputum, stool, urine, cerebrospinal fluid, tissue embedded in paraffin, for example, tissue from eyes, intestine, kidney, brain, heart, prostate, lungs, breast or liver, histological slides and all possible combinations thereof.

A method is also preferred for the diagnosis and/or prognosis of adverse events for patients or individuals, whereby these adverse events are associated with methylation patterns within the MHC. The subject of the present invention is also the use of a method according to the invention, wherein significant genetic parameters are diagnosed within the MHC. Finally, another subject of the present invention is a kit comprised of a bisulfite-containing reagent, sets of primers according to the invention for the production of amplified products, oligonucleotides and/or PNA oligomers according to the invention, as well as instructions for conducting and evaluating a method according to the invention.

The present invention thus describes a set of at least 10 oligomer probes (oligonucleotides and/or PNA oligomers), which serve for the detection of the state of cytosine methyation in chemically pretreated genomic DNA (SEQ ID-NO:1 or SEQ ID-NO:2). The diagnosis of genetic and epigenetic parameters within the major histocompatibility complex (MHC) is possible with these probes. In addition, a method is described, which is specific for the diagnosis of genetic and epigenetic parameters within the MHC.

Segments of SEQ ID-NO.1 or SEQ ID-NO:2 that are at least 20 base pairs long of the above-named chemically pretreated DNA are utilized for diagnosis. Oligonucleotides with a length of 13 nucleotides that are either inversely complementary or identical or PNA oligomers with a length of 9 nucleotides that are also either inversely complementary or identical are used as detectors for these [pretreated genomic] segments.

Both the oligonucleotides as well as the PNA oligomers contain at least one CpG dinucleotide. The cytosine of the respective CpG dinucleotide is the 5th to the 9th nucleotide as viewed from the 5′ end of the oligonucleotide. The cytosine of the CpG dinucleotide, in contrast, is the 4th to the 6th nucleotide considered from the 5′ end of the PNA oligomer. It is a decisive factor that an oligonucleotide from SEQ ID-NO:1 or SEQ ID-NO:2 is present for each of the CpG dinucleotides in the respective set of oligonucleotides or PNA oligomers.

It is also important in this respect that one must not analyze individual CpG dinucleotides, but rather the plurality of CpG dinucleotides present in the sequences for the diagnosis of genetic parameters within the MHC. In a particularly preferred variant of the method, all CpG dinucleotides present in the sequences are to be investigated.

In a preferred variant of the method according to the invention, the oligonucleotides or PNA oligomers are bound to defined sites on a solid phase.

It is preferred that different amplified products are arranged on the planar solid phase in the form of a rectangular or hexagonal lattice.

The nucleic acids, oligonucleotides or PNA oligomers are preferably used for the diagnosis of rheumatoid arthritis, diabetes, hereditary hemochromatosis, schizophrenia, multiple sclerosis, systemic lupus erythematosus, sarcoidosis, cirrhosis, myositis, psoriasis, nephritis, cancer, particularly head or neck cancer, IgA nephropathy, hypertension, Behcet's disease, GeeHeubner-Herter-Thaysen disease, myasthenia gravis, spondyloarthropathy, tuberculosis, hypertrophic cardiomyopathy, Basedow's [Graves'] disease, juvenile chronic arthritis, epilepsy, idiopathic generalized epilepsy or juvenile myoclonic epilepsy, Takayasu disease, multiple immunopathological diseases, for susceptibility to leprosy, for susceptibility to malaria and/or for susceptibility to leishmaniasis, by analyis of methylation patterns within the MHC.

Also, the nucleic acids according to SEQ ID-NO:1 or SEQ ID-NO:2, which are listed in the Appendix, are preferably used for the diagnosis of genetic and epigenetic parameters within the major histocompatibility complex (MHC).

In addition, a method for the diagnosis of important genetic parameters within the MHC by analysis of cytosine methylations and single nucleotide polymorphisms in genomic DNA samples is described. For this purpose, one proceeds by the following steps:

In the first step of the method, a genomic DNA sample is chemically treated such that cytosine bases that are not methylated in the 5′ position are converted to uracil, thymine or another base unlike cytosine in its hybridization behavior.

The genomic DNA to be analyzed is preferably obtained from the usual sources for DNA, such as, e.g., cell lines, blood, sputum, stool, urine, cerebrospinal fluid, tissue embedded in paraffin, for example, tissue from eyes, intestine, kidney, brain, heart, prostate, lungs, breast or liver, histological slides and all possible combinations thereof. The above-described treatment of genomic DNA with bisulfite (hydrogen sulfite, disulfite) and subsequent alkaline hydrolysis, which leads to a conversion of unmethylated cytosine nucleobases to uracil is preferred for this purpose.

In the second step of the method, fragments of the chemically pretreated genomic DNA are amplified with the use of primer oligonucleotides.

More than 10 different fragments, which are 100-2000 base pairs long, are preferred.

In a preferred variant of the method, the amplification is conducted by means of the polymerase chain reaction (PCR), in which a heat-stable DNA polymerase is preferably used.

It is preferred according to the invention that the amplification of several DNA segments is conducted in one reaction vessel.

In a preferred variant of the method, the set of primer oligonucleotides comprises at least two oligonucleotides, each of which has a sequence that is at least 18 base pairs long and that is identical to or is inversely complementary to a segment of the base sequences according to SEQ ID-NO:1 or SEQ ID-NO:2. The primer oligonucleotides are preferably characterized in that they do not contain a CpG dinucleotide.

It is preferred according to the invention that at least one primer is bound to a solid phase in the amplification. According to the invention it is further preferred that different oligonucleotide and/or PNA oligomer sequences are arranged on a planar solid phase in the form of a rectangular or hexagonal lattice.

The solid-phase surface is preferably comprised of silicon, glass, polystyrene, aluminum, steel, iron, copper, nickel, silver, or gold.

In the third step of the method, the amplified products are hybridized to a set of at least 10 oligonucleotide or PNA oligomer probes.

The given oligonucleotides comprise at least one base sequence with a length of 13 nucleotides, which is inversely complementary or identical to a segment of the base sequences listed in the Appendix, which contains at least one CpG dinucleotide. The cytosine of the CpG dinucleotide is the 5th to 9th nucleotide viewed from the 5′ end of the 13-mer. An oligonucleotide is present for each CpG dinucleotide.

The given PNA oligomers comprise at least one base sequence with a length of 9 nucleotides, which is inversely complementary or identical to a segment of the base sequences according to SEQ ID-NO:1 or SEQ ID-NO:2, which contains at least one CpG dinucleotide. The cytosine of the CpG dinucleotide is the 4th to 6th nucleotide viewed from the 5′ end of the 9-mer. An oligonucleotide^{★}is present for each CpG dinucleotide. ^{★}sic; PNA oligomer?—Trans note.

In the fourth step of the method, the non-hybridized amplified products are removed.

In the last step of the method, the hybridized amplified products are detected.

It is preferred according to the invention that labels that are introduced on the amplified products at each position of the solid phase where an oligonucleotide sequence is found can be identified.

It is preferred according to the invention that the labels of the amplified products are fluorescence labels.

It is preferred according to the invention that the labels of the amplified products are radionuclides.

It is preferred according to the invention that the labels of the amplified products are removable molecular fragments with typical mass, which are detected in a mass spectrometer.

According to the invention it is preferred that the amplified products, fragments of the amplified products or probes that are complementary to the amplified products, are detected in the mass spectrometer.

It is preferred according to the invention that the generated fragments have a single positive or negative net charge for better detectability in the mass spectrometer.

It is preferred according to the invention that detection and visualization are conducted by means of matrix-assisted laser desorption/ionization mass spectrometry (MALDI) or by means of electrospray mass spectrometry (ESI).

A method is also preferred for the diagnosis and/or prognosis of adverse events for patients or individuals, whereby these adverse events are associated with important genetic parameters within the MHC.

According to the invention, the use of a method for the diagnosis of important genetic parameters within the MHC is preferred.

The subject of the present invention is also a kit comprising a bisulfite-containing reagent, a set of primer oligonucleotides comprising at least two oligonucleotides, each of whose sequence is a segment at least 18 base pairs long, and [these oligonucleotides] correspond to or are complmentary to the base sequences according to SEQ ID-NO:1 or SEQ ID-NO:2 for the production of amplified products, oligonucleotides and/or PNA oligomers, as well as instructions for conducting and evaluating the described method.

The following example concerns a fragment of the HLA-A gene, in which a specific CG position is investigated for methylation.

In the first step, a genomic sequence is converted with the use of bisulfite (hydrogen sulfite, disulfite) and subsequent alkaline hydrolysis. This converted DNA serves for the purpose of detecting methylated cytosines. In the present case, the cytosines of the HLA-A gene of the length of 3201 bases are investigated. For this purpose, a defined fragment of length 874 is amplified with the specific primers TTTGGTTTTGATTTAGATTTGG and AAATAAACTCTCTAACTACTC. This amplified product serves as the sample, which is hybridized to an oligonucleotide that has previously been bound to a solid phase, for example, TAGGTCGTTTATA, whereby the cytosine to be detected is found at position 487 of the amplified product. The detection of the hybridization product is based on primers fluorescently labeled with Cy3 and Cy5, which were used for the amplification. A hybridization reaction of the amplified DNA with the oligonucleotide occurs only if a methylated cytosine has been present in this place in the bisulfite-treated DNA. Thus the methylation state of the respective cytosine to be investigated is decisive for the hybridization product.

The following examples explain the invention:

### EXAMPLE 1

### Conducting the Methylation Analysis of the DAXX Gene Localized in the MHC

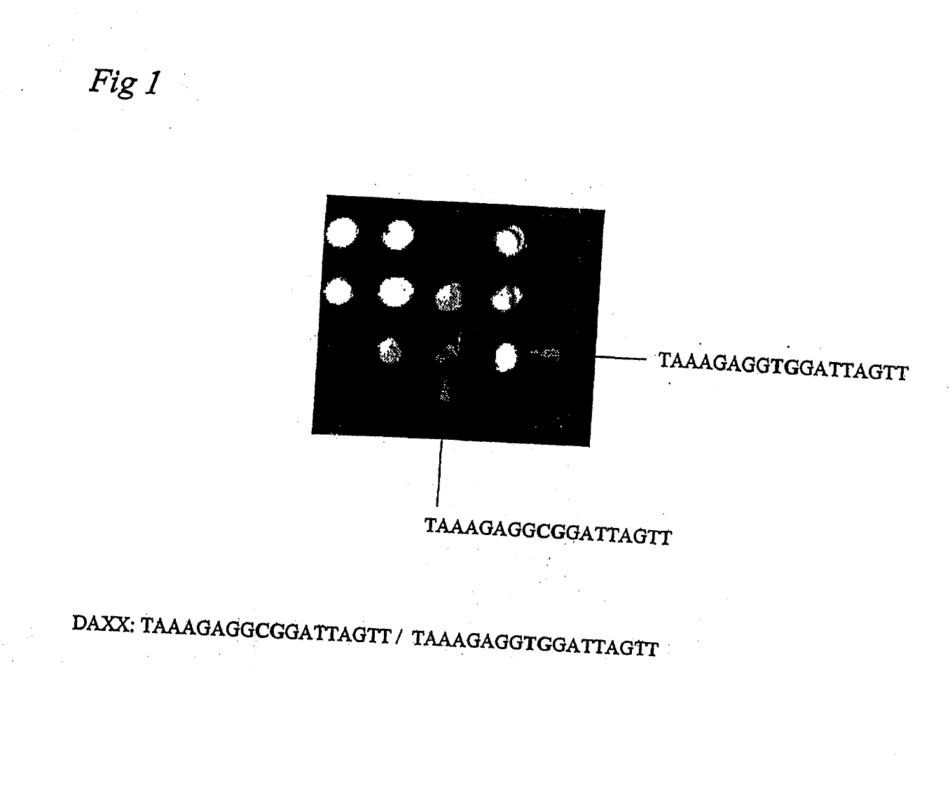

The following example (FIG. 1) concerns a fragment of the DAXX gene, in which a specific CG position is investigated for methylation.

In the first step, a genomic sequence is treated with the use of bisulfite (hydrogen sulfite, disulfite) in such a way that all of the cytosines not methylated at the 5-position of the base are modified such that a base that is different with respect to its base pairing behavior is formed, whereas the cytosines that are methylated in the 5-position remain unchanged. If bisulfite is used for the reaction, then an addition occurs on the unmethylated cytosine bases. Also, a denaturing reagent or solvent as well as a radical trap must be present. A subsequent alkaline hydrolysis then leads to the conversion of unmethylated cytosine nucleobases to uracil. This converted DNA serves for the purpose of detecting methylated cytosines. In the second step of the method, the treated DNA sample is diluted with water or an aqueous solution. Preferably, a DNA desulfonation is then conducted. In the third step of the method, the DNA sample is amplified in a polymerase chain reaction, preferably with a heat-stable DNA polymerase. In the present case, cytosines of the DAXX gene are investigated. For this purpose, a defined fragment with a length of 880 bp is amplified with the specific primer oligonucleotides TTAGGTTTTGGTTTGTTGATGAG and CCCTAACTCCTCTAAACCTCA. This amplified product serves as the sample, which hybridizes to an oligonucleotide that has previously been bound to a solid phase, for example, TAAAGAGGCGGATTAGTT, with the formation of a duplex structure, whereby the cytosine to be detected is found at position 142 of the amplified product. The detection of the hybridization product is based on primer oligonucleotides fluorescently labeled with Cy3 and Cy5, which were used for the amplification. A hybridization reaction of the amplified DNA with the oligonucleotide occurs only if a methylated cytosine has been present in this place in the bisulfite-treated DNA. Thus the methylation state of the respective cytosine to be investigated is decisive for the hybridization product. In the present case, an unmethylated state is detected for the oligomer.

### EXAMPLE 2

### Conducting the Methylation Analysis of the RXRB Gene Localized in the MHC

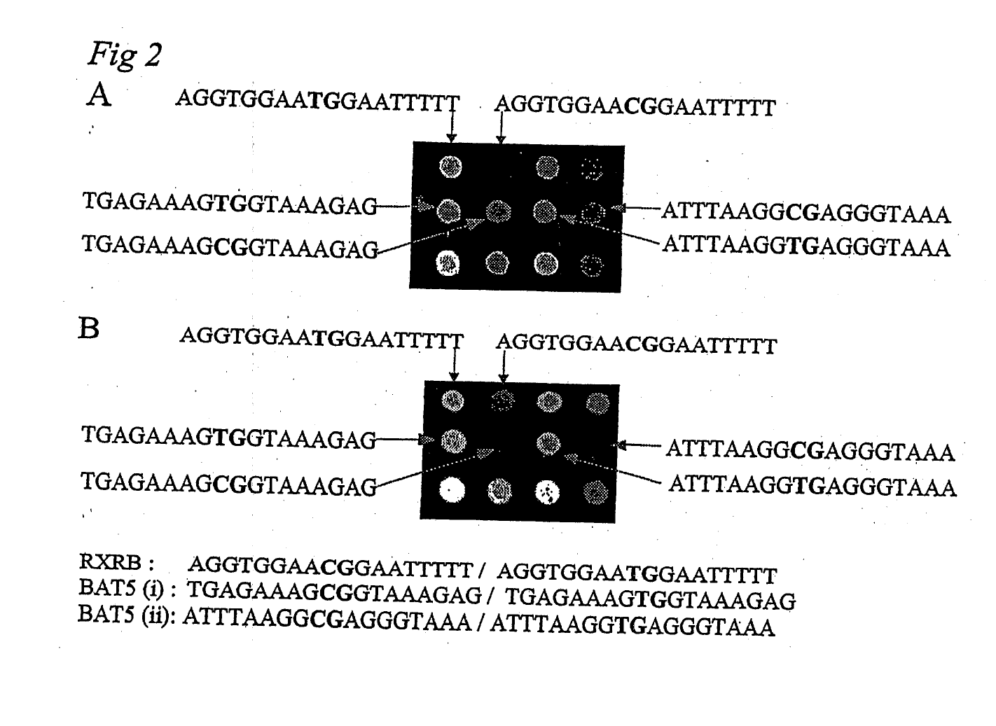

The following example (FIG. 2) concerns a fragment of the RXRB gene, in which a specific CG position is investigated for methylation.

In the first step, a genomic sequence is treated with the use of bisulfite (hydrogen sulfite, disulfite) in such a way that all of the cytosines not methylated at the 5-position of the base are modified such that a base that is different with respect to its base pairing behavior is formed, whereas the cytosines that are methylated in the 5-position remain unchanged. If bisulfite is used for the reaction, then an addition occurs on the unmethylated cytosine bases. Also, a denaturing reagent or solvent as well as a radical trap must be present. A subsequent alkaline hydrolysis then leads to the conversion of unmethylated cytosine nucleobases to uracil. This converted DNA serves for the purpose of detecting methylated cytosines. In the second step of the method, the treated DNA sample is diluted with water or an aqueous solution. Preferably, a DNA desulfonation is then conducted. In the third step of the method, the DNA sample is amplified in a polymerase chain reaction, preferably with a heat-stable DNA polymerase. In the present case, cytosines of the RXRB gene are investigated. For this purpose, a defined fragment with a length of 385 bp is amplified with the specific primer oligonucleotides ATATTGGTAAAGGTATTAGGG and ACTTAACTCAACTCTATACCTAC. This amplified product serves as the sample, which hybridizes to an oligonucleotide that has previously been bound to a solid phase, for example, AGGTGGAACGGAATTTTT, with the formation of a duplex structure, whereby the cytosine to be detected is found at position 33 of the amplified product. The detection of the hybridization product is based on primer oligonucleotides fluorescently labeled with Cy3 and Cy5, which were used for the amplification. A hybridization reaction of the amplified DNA with the oligonucleotide occurs only if a methylated cytosine has been present in this place in the bisulfite-treated DNA. Thus the methylation state of the respective cytosine to be investigated is decisive for the hybridization product. In the present case, an unmethylated state is detected for the oligomer in illustration A and a partially methylated state is detected in illustration B.

### EXAMPLE 3

### Conducting the Methylation Analysis of the BAT5 Gene Localized in the MHC

The following example (FIG. 2^{★}) concerns a fragment of the BAT5 gene, in which a specific CG position is investigated for methylation. ^{★}sic—FIG. 3?—Trans. Note.

In the first step, a genomic sequence is treated with the use of bisulfite (hydrogen sulfite, disulfite) in such a way that all of the cytosines not methylated at the 5-position of the base are modified such that a base that is different with respect to its base pairing behavior is formed, whereas the cytosines that are methylated in the 5-position remain unchanged. If bisulfite is used for the reaction, then an addition occurs on the unmethylated cytosine bases. Also, a denaturing reagent or solvent as well as a radical trap must be present. A subsequent alkaline hydrolysis then leads to the conversion of unmethylated cytosine nucleobases to uracil. This converted DNA serves for the purpose of detecting methylated cytosines. In the second step of the method, the treated DNA sample is diluted with water or an aqueous solution. Preferably, a DNA desulfonation is then conducted. In the third step of the method, the DNA sample is amplified in a polymerase chain reaction, preferably with a heat-stable DNA polymerase. In the present case, cytosines of the BAT5 gene are investigated. For this purpose, a defined fragment with a length of 539 bp is amplified with the specific primer oligonucleotides AGAAGAGAATGTGGGTAGGA and AAAACCTACTTATCAAACCAAT. This amplified product serves as the sample, which hybridizes to an oligonucleotide that has been previously bound to a solid phase, for example i) TGAGAAAGCGGTAAAGAG or ii) ATTTAAGGCGAGGGTAAA, with the formation of a duplex structure, whereby the cytosine to be detected is found at position 211 for the oligomer TGAGAAAGCGGTAAAGAG or at position 431 for the oligomer ATTTAAGGCGAGGGTAAA. The detection of the hybridization product is based on primer oligonucleotides fluorescently labeled with Cy3 and Cy5, which were used for the amplification. A hybridization reaction of the amplified DNA with the oligonucleotide occurs only if a methylated cytosine has been present in this place in the bisulfite-treated DNA. Thus the methylation state of the respective cytosine to be investigated is decisive for the hybridization product. In the present case, a partially methylated state is detected for both oligomers in illustration A and an unmethylated state is detected for each in illustration B.

1. Nucleic acids for the diagnosis of a set of genetic parameters within the major histocompatibility complex (MHC), comprising a segment which is at least 20 base pairs long that is inversley complementary or identical to the chemically pretreated genomic DNA according to SEQ ID-NO:1 or SEQ ID-NO:2.

2. Oligonucleotides for the detection of the cytosine methylation state in pretreated genomic DNA, each comprising at least one base sequence with a length of at least 13 nucleotides, which is inversely complementary or identical to a segment of the base sequences according to SEQ ID-NO:1 or SEQ ID-NO:2, which contains at least one CpG dinucleotide.

3. The oligonucleotide according to claim 2, further characterized in that the cytosine of the CpG dinucleotide is the 5^{th} to the 9^{th} nucleotide from the 5′ end of the 13-mer.

4. A set of oligonucleotides according to claim 2, characterized in that an oligonucleotide is present for each of the CpG dinucleotides from one of [the sequences] SEQ ID-NO:1 or SEQ ID-NO:2.

5. PNA (peptide nucleic acid) oligomers for the detection of the cytosine methylation state in chemically pretreated genomic DNA, each comprising at least one PNA base sequence with a length of at least 9 nucleotides, which is inversely complementary or identical to a segment of the base sequences according to SEQ ID-NO:1 or SEQ ID-NO:2, which contains at least one CpG dinucleotide.

6. PNA oligomers according to claim 5, further characterized in that the cytosine of the CpG dinucleotide is the 4^{th} to the 6^{th} nucleotide from the 5′ end of the 9-mer.

7. A set of PNA oligomers according to claim 5, characterized in that an oligonucleotide is present for each of the CpG dinucleotides from one base sequence according to SEQ ID-NO:1 or SEQ ID-NO:2.

8. A set of oligomer probes for the detection of the cytosine methylation state and/or of single nucleotide polymorphisms in chemically pretreated genomic DNA, comprising at least 10 of the oligonucleotides or PNA sequences of claims 2 to 7.

9. An arrangement of different oligonucleotides and/or PNA oligomer sequences according to one of claims 2 to 8, characterized in that these sequences-are bound to defined sites of a solid phase.

10. The arrangement of different oligonucleotides and/or PNA oligomer sequences according to claim 9, further characterized in that these sequences are arranged on a planar solid phase in the form of a rectangular or hexagonal lattice.

11. A set of primer oligonucleotides comprising at least two oligonucleotides, each of which has a sequence that corresponds to or is inversely complementary to the base sequences according to SEQ ID-NO:1 or SEQ ID-NO:2 and is at least 18 base pairs long.

12. The set of primer oligonucleotides according to claim 11, further characterized in that these do not contain a CpG dinucleotide.

13. The set of primer oligonucleotides according to claim 11 or 12, further characterized in that at least one primer is bound to a solid phase.

14. Use of nucleic acids, oligonucleotides or PNA oligomers according to one of claims 1 to 13 for the diagnosis of autoimmune disorders by analysis of methylation patterns within the MHC.

15. Use of nucleic acids, oligonucleotides or PNA-oligomers according to one of claims 1 to 13 for the diagnosis of rheumatoid arthritis by analysis of methylation patterns within the MHC.

16. Use of nucleic acids, oligonucleotides or PNA oligomers according to one of claims 1 to 13 for the diagnosis of diabetes by analysis of methylation patterns within the MHC.

17. Use of nucleic acids, oligonucleotides or PNA oligomers according to claim 16, further characterized in that the diabetes involves type I diabetes or insulin-dependent diabetes mellitus (IDDM).

18. Use of nucleic acids, oligonucleotides or PNA oligomers according to one of claims 1 to 13 for the diagnosis of hereditary hemochromatosis by analysis of methylation patterns within the MHC.

19. Use of nucleic acids, oligonucleotides or PNA oligomers according to claim 18, further characterized in that the the hereditary hemochromatosis involves genetic hemochromatosis (GH) or the mild form of hemochromatosis.

20. Use of nucleic acids, oligonucleotides or PNA oligomers according to one of claims 1 to 13 for the diagnosis of schizophrenia by analysis of methylation patterns within the MHC.

21. Use of nucleic acids, oligonucleotides or PNA oligomers according to one of claims 1 to 13 for the diagnosis of multiple sclerosis by analysis of methylation patterns within the MHC.

22. Use of nucleic acids, oligonucleotides or PNA oligomers according to one of claims 1 to 13 for the diagnosis of systemic lupus erythematosus by analysis of methylation patterns within the MHC.

23. Use of nucleic acids, oligonucleotides or PNA oligomers according to one of claims 1 to 13 for the diagnosis of sarcoidosis by analysis of methylation patterns within the MHC.

24. Use of nucleic acids, oligonucleotides or PNA oligomers according to one of claims 1 to 13 for the diagnosis of primary biliary cirrhosis by analysis of methylation patterns within the MHC.

25. Use of nucleic acids, oligonucleotides or PNA oligomers according to one of claims 1 to 13 for the diagnosis of myositis by analysis of methylation patterns within the MHC.

26. Use of nucleic acids, oligonucleotides or PNA oligomers according to one of claims 1 to 13 for the diagnosis of psoriasis by analysis of methylation patterns within the MHC.

27. Use of nucleic acids, oligonucleotides or PNA oligomers according to one of claims 1 to 13 for the diagnosis of nephritis by analysis of methylation patterns within the MHC.

28. Use of nucleic acids, oligonucleotides or PNA oligomers according to one of claims 1 to 13 for the diagnosis of cancer by analysis of methylation patterns within the MHC.

29. Use of nucleic acids, oligonucleotides or PNA oligomers according to claim 28, further characterized in that the types of cancer involve head or neck cancer.

30. Use of nucleic acids, oligonucleotides or PNA oligomers according to one of claims 1 to 13 for the diagnosis of IgA nephropathy by analysis of methylation patterns within the MHC.

31. Use of nucleic acids, oligonucleotides or PNA oligomers according to one of claims 1 to 13 for the diagnosis of hypertension by analysis of methylation patterns within the MHC.

32. Use of nucleic acids, oligonucleotides or PNA oligomers according to one of claims 1 to 13 for the diagnosis of Behcet's disease by analysis of methylation patterns within the MHC.

33. Use of nucleic acids, oligonucleotides or PNA oligomers according to one of claims 1 to 13 for the diagnosis of Gee-Heubner-Herter-Thaysen disease (coeliac disease) by analysis of methylation patterns within the MHC.

34. Use of nucleic acids, oligonucleotides or PNA oligomers according to one of claims 1 to 13 for the diagnosis of myasthenia gravis by analysis of methylation patterns within the MHC.

35. Use of nucleic acids, oligonucleotides or PNA oligomers according to one of claims 1 to 13 for the diagnosis of spondyloarthropathy by analysis of methylation patterns within the MHC.

36. Use of nucleic acids, oligonucleotides or PNA oligomers according to one of claims 1 to 13 for the diagnosis of tuberculosis by analysis of methylation patterns within the MHC.

37. Use of nucleic acids, oligonucleotides or PNA oligomers according to one of claims 1 to 13 for the diagnosis of hypertrophic cardiomyopathy by analysis of methylation patterns within the MHC.

38. Use of nucleic acids, oligonucleotides or PNA oligomers according to one of claims 1 to 13 for the diagnosis of Basedow's [Graves'] disease by analysis of methylation patterns within the MHC.

39. Use of nucleic acids, oligonucleotides or PNA oligomers according to one of claims 1 to 13 for the diagnosis of juvenile rheumatoid arthritis by analysis of methylation patterns within the MHC.

40. Use of nucleic acids, oligonucleotides or PNA oligomers according to one of claims 1 to 13 for the diagnosis of epilepsy by analysis of methylation patterns within the MHC.

41. Use of nucleic acids, oligonucleotides or PNA oligomers according to claim 40, further characterized in that the types of epilepsy involve idiopathic generalized epilepsy or juvenile myoclonic epilepsy.

42. Use of nucleic acids, oligonucleotides or PNA oligomers according to one of claims 1 to 13 for the diagnosis of Takayasu disease by analysis of methylation patterns within the MHC.

43. Use of nucleic acids, oligonucleotides or PNA oligomers according to one of claims 1 to 13 for the diagnosis of multiple immunopathological diseases by analysis of methylation patterns within the MHC.

44. Use of nucleic acids, oligonucleotides or PNA oligomers according to one of claims 1 to 13 for the diagnosis of susceptibility to leprosy by analysis of methylation patterns within the MHC.

45. Use of nucleic acids, oligonucleotides or PNA oligomers according to one of claims 1 to 13 for the diagnosis of susceptibility to malaria by analysis of methylation patterns within the MHC.

46. Use of nucleic acids, oligonucleotides or PNA oligomers according to one of claims 1 to 13 for the diagnosis of susceptibility to leishmaniasis by analysis of methylation patterns within the MHC.

47. Use of nucleic acids according to claim 1 for the diagnosis of important genetic parameters within the MHC.

48. A method for the diagnosis of important genetic parameters within the MHC by analysis of cytosine methylations in sets of oligonucleotides or PNA oligomers according to one of the preceding claims, characterized in that the following steps are conducted:
a) In a genomic DNA sample, cytosine bases that are not methylated at the 5-position are converted by chemical treatment to uracil, thymidine or another base that is unlike cytosine in its hybridization behavior;
b) fragments of this chemically pretreated genomic DNA are amplified with the use of sets of primer oligonucleotides according to claim 11 or 12 and a polymerase;
c) the amplified products are hybridized to a set of oligonucleotide or PNA probes of claims 2 to 8;
d) the hybridized amplified products are detected and visualized.

49. The method according to claim 48, further characterized in that more than ten different fragments, which are 100-2000 base pairs long, are amplified.

50. The method according to claim 48, further characterized in that the chemical treatment is conducted by means of a solution of a bisulfite, hydrogen sulfite or disulfite.

51. The method according to claim 48, further characterized in that the polymerase is a heat-stable DNA polymerase.

52. The method according to claims 48-51, further characterized in that the amplification is conducted by means of the polymerase chain reaction (PCR).

53. The method according to one of the preceding claims 48-52, further characterized in that the labels introduced on the amplified products can be identified at any position of the solid phase where an oligonucleotide sequence is found.

54. The method according to claim 53, further characterized in that that an arrangement according to claim 9 or 10 is used and that the solid-phase surface is comprised of silicon, glass, polystyrene, aluminum, steel, iron, copper, nickel, silver or gold.

55. The method according to one of the preceding claims 48-54, further characterized in that the amplification of several DNA segments is conducted in one reaction vessel.

56. The method according to claim 53, further characterized in that the labels of the amplified products are fluorescence labels.

57. The method according to claim 53, further characterized in that the labels of the amplified products are radionuclides.

58. The method according to claim 53, further characterized in that the labels of the amplified products are removable molecular fragments with typical mass, which can be detected in a mass spectrometer.

59. The method according to one of claims 48, 49 or 53, further characterized in that the amplified products or fragments of the amplified products are detected in the mass spectrometer.

60. The method according to claim 58 or 59, further characterized in that the generated fragments have a single positive or negative net charge for better detectability in the mass spectrometer.

61. The method according to one of claims 58 to 60, further characterized in that detection and visualization are conducted by means of matrix-assisted laser desorption/ionization mass spectrometry (MALDI) or by means of electrospray mass spectrometry (ESI).

62. The method according to one of the preceding claims 48 to 51, wherein the genomic DNA has been obtained from a DNA sample, whereby sources for DNA include, e.g., cell lines, biopsies, blood, sputum, stool, urine, cerebrospinal fluid, tissue embedded in paraffin, for example, tissue from eyes, intestine, kidney, brain, heart, prostate, lungs, breast or liver, histological slides and all possible combinations thereof.

63. The method according to one of the preceding claims 48 to 62 for the diagnosis and/or prognosis of adverse events for patients or individuals, whereby these adverse events are associated with methylation patterns within the MHC.

64. Use of a method according to one of claims 48 to 63, characterized in that important genetic parameters are diagnosed within the MHC.

65. A kit comprising a bisulfite-containing reagent, sets of primers according to claim 11 or 12 for the production of amplified products, oligonucleotides and/or PNA oligomers according to one of claims 2 to 8 as well as instructions for conducting and evaluating a method according to one of claims 48 to 63.
